(19) United States
(12) Patent Application Publication
(10) Pub. No.: US 2014/0324126 A1
(43) Pub. Date: Oct. 30, 2014

(54) Heating Control for an Inductive External Charger for an Implantable Medical Device

(71) Applicant: Boston Scientific Neuromodulation Corporation, Valencia, CA (US)

(72) Inventor: Robert Ozawa, Woodland Hills, CA (US)

(73) Assignee: Boston Scientific Neuromodulation Corporation, Valencia, CA (US)

(21) Appl. No.: 14/182,068

(22) Filed: Feb. 17, 2014

Related U.S. Application Data
(60) Provisional application No. 61/816,427, filed on Apr. 26, 2013.

(51) Int. Cl. A61N 1/378; A61N 1/36

(52) U.S. Cl. CPC A61N 1/3787; A61N 1/36125
USPC 607/60

(57) ABSTRACT

The disclosed technique for charging a battery in an implantable medical device using an external charger indirectly determines the total power dissipated as heat in the IPG (P_IPG) by accounting for the various powers in the external charger/IPG system which are either known or can be measured, such as the input power provided to the amplifier that drives the coil in the external charger (Psys), the power stored in the IPG's battery (Pstored), and the power dissipated in the external charger's charging coil as heat (P_EC) (which is measured). Determining P_IPG at the external charger in this manner allows the heat flux from the IPG to be calculated (F_IPG), and compared to a safe heat flux limit (F_IPG′) to allow for adjustment to the power of the magnetic charging field in a closed loop fashion.

### CROSS REFERENCE TO RELATED APPLICATIONS

This is a non-provisional application of U.S. Provisional Patent Application Ser. No. 61/816,427, filed Apr. 26, 2013, which is incorporated herein by reference, and to which priority is claimed.

### FIELD OF THE INVENTION

The present invention relates to wireless battery charging in implantable medical device systems.

### BACKGROUND

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system.

As shown in FIGS. 1A-1C, a SCS system typically includes an Implantable Pulse Generator (IPG) 10, which includes a biocompatible device case 12 formed of a conductive material such as titanium for example. The case 12 typically holds the circuitry and battery 14 necessary for the IPG to function, although IPGs can also be powered via external RF energy and without a battery. The IPG 10 is coupled to electrodes 16 via one or more electrode leads (two such leads 18 and 20 are shown), such that the electrodes 16 form an electrode array 22. The electrodes 16 are carried on a flexible body 24, which also houses the individual signal wires 26 coupled to each electrode. In the illustrated embodiment, there are eight electrodes on lead 18, labeled E₁-E₈, and eight electrodes on lead 20, labeled E₉-E₁₆, although the number of leads and electrodes is application specific and therefore can vary. The leads 18, 20 couple to the IPG 10 using lead connectors 28, which are fixed in a non-conductive header material 30, which can comprise an epoxy for example.

As shown in the cross-section of FIG. 1C, the IPG 10 typically includes an electronic substrate assembly including a printed circuit board (PCB) 32, along with various electronic components 34 mounted to the PCB. Two coils (more generally, antennas) are generally present in the IPG 10: a telemetry coil 36 used to transmit/receive data to/from an external controller (not shown); and a charging coil 38 for charging or recharging the IPG's battery 14 using an external charger 50 (discussed further below). In this example, the telemetry coil 36 and charging coil 38 are within the case 12, as disclosed in U.S. Patent Publication 2011/0112610. (FIG. 1B shows the IPG 100 with the case 12 removed to ease the viewing of the two coils 36 and 38).

FIG. 2 shows the IPG 10 in communication with external charger 50 just mentioned. The external charger 50 is used to wirelessly convey power 90 to the IPG 10 in the form of an AC magnetic charging field used to recharge the IPG's battery 14. The transfer of power from the external charger 50 is enabled by a coil (antenna) 52. The external charger 50, like the IPG 100, also contains a PCB 54 on which electronic components 56 are placed. Some of these electronic components 56 are discussed subsequently. A user interface 58, including touchable buttons and perhaps a display and a speaker, allows a patient or clinician to operate the external charger 50. A battery 60 provides power for the external charger 50, which battery may itself be rechargeable. The external charger 50 can also receive AC power from a wall plug. A hand-holdable case 62 sized to fit a user's hand contains all of the external charger 50's components.

Power transmission from the external charger 50 to the IPG 10 occurs wirelessly, and transcutaneously through a patient's tissue 25, via inductive coupling. FIG. 3 shows details of the circuitry used to implement such functionality. Coil 52 in the external charger 50 is energized via a driver circuit (e.g., an amplifier 74) with a constant non-data-modulated AC current, Icharge, to create the charging field 90. The charging field 90 induces a current in the charging coil 38 within the IPG 10, which current is rectified (82) to DC levels, and used to recharge the battery 14, perhaps via a charging and battery protection circuit 84 as shown. Although not shown, one skilled will understand that each of coils 52 and 38 are associated with a capacitor to form a tank circuit, which capacitor may be either in series or in parallel with the coils. The frequency of the magnetic charging field 90 can be perhaps 80 kHz or so. When charging the battery 14 in this manner, is it typical that the case 62 of the external charger 50 touches the patient's tissue 25, although this is not strictly necessary.

The IPG 10 can also telemeter data 92 back to the external charger 50 during a charging session using reflected impedance modulation, which is sometimes known in the art as Load Shift Keying (LSK). Such back telemetry 92 from the IPG 10 can provide useful data concerning charging to the external charger 50, such as the capacity of the battery 14, or whether charging is complete and the external charger 50 can cease. Further details concerning LSK telemetry can be found in U.S. patent application Ser. No. 13/608,600, filed Sep. 10, 2012. Briefly, digital LSK data to be transmitted is formed in an LSK module 86, associated with the control circuit 80 in the IPG 10. This data opens or closes transistors 96, which shorts the IPG's charging coil 38 to modulate its impedance. This impedance modulation varies the voltage across the charging coil 52 used to produce the charging field 90, and such voltage variations are interpreted by demodulator 72 to recover the LSK data. Other more traditional means of telemetry can also be used to communicate between the external charge 50 and the IPG 10.

It is generally desirable to charge the IPG's battery 14 as quickly as possible to minimize inconvenience to the patient. One way to decrease charging time is to increase the strength of the magnetic charging field 90 by increasing Icharge in the coil 52 of the external charger 50. Increasing the charging field 90 will increase the current/voltage induced in the coil 38 of the IPG 100, which increases the battery charging current, Ibat2. However, the strength of the magnetic charging field can only be increased so far before heating becomes a concern. Heating is an inevitable side effect of inductive charging using magnetic fields, and can result from the circuitry energized during the charging process, or eddy currents formed in conductive structures in both the external charger 50 and the IPG 10. Heating is a serious safety concern. The external charger 50 as mentioned is usually in contact with the patient's tissue, and of course the IPG 10 is inside the patient. If the temperature of either exceeds a given safe temperature, the patient's tissue may be aggravated or damaged.

The art has recognized that heating can be monitored in either or both of the external charger 50 and IPG 10 using temperature sensors such as thermistors, and by controlling the charging field 90 based on the detected temperature(s). For example, if control circuitry 70 (e.g., a microcontroller) in the external charger 50 understands a reported temperature to be too high, it can control the driver circuit 74 to either reduce Icharge, or its duty cycle (i.e., the percentage of time that the magnetic charge field is actually produced), or both, until a safe temperature is reached for either or both of the devices. See, e.g., U.S. Patent Application Publication 2011/0087307.

The external charger 50 can also be made aware of certain electrical parameters during charging that can affect heat. For example, in the above-mentioned '307 Publication, the IPG 10 can periodically telemeter electrical parameters indicative of heating to the external charger during a charging session. For example, the IPG 10 can telemeter information about the battery charging current, Ibat2, and/or other parameters indicative of the battery charging current, such as the voltage drop across charging and protection circuitry 134. Such electrical parameters are generally indicative of heat generation, and so are useful in adjusting the power of the charging field 90, as the '307 Publication explains.

### BRIEF DESCRIPTION OF THE DRAWINGS

FIGS. 1A-1C show different views of an implantable medical device, specifically an Implantable Pulse Generator (IPG) in accordance with the prior art.

FIG. 2 shows wireless links between the IPG and an external charger in accordance with the prior art.

FIG. 3 shows circuitry in both the IPG and external charger for providing power to the IPG in accordance with the prior art.

FIGS. 4A and 4B show different orientations between the external charger and the IPG, and their effects on heat dissipation in the IPG.

FIG. 5 shows an improved external charger including an algorithm for controlling charging based on a determined heat flux of the IPG.

FIG. 6 shows further details of the circuitry in the improved external charger and in the IPG.

FIG. 7 shows the various powers input to and dissipated by the external charger/IPG system, and shows how IPG power dissipation and heat flux can be calculated.

FIGS. 8 and 9 show different algorithms used for charging the IPG in accordance with the determined IPG heat flux.

### DETAILED DESCRIPTION

The inventor has realized that prior approaches to determining and controlling heating when inductively charging a battery in an IPG have shortcomings. Solutions that require thermocouples to measure temperatures during charging measure heat at only discrete locations, which may not be indicative of the total amount of heat that is being dissipated by the IPG.

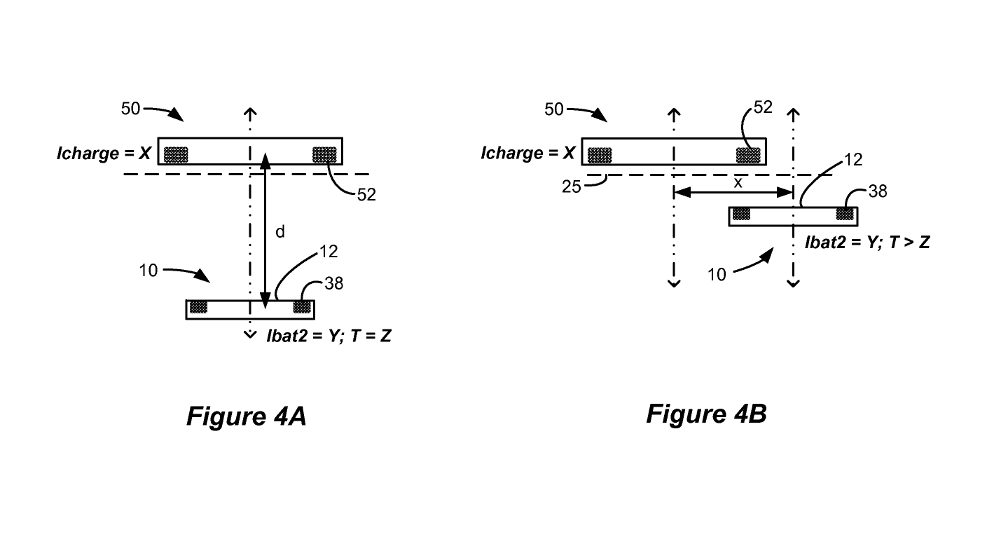

Solutions relying on telemetry of electrical parameters from the IPG, such as discussed above with respect to the '307 Publication, essentially inform the external charger only of the rate at which the IPG's battery is charging. While such charging rate is generally indicative of heating, and therefore useful, it is not indicative of the total heat dissipated by the IPG, which can vary depending on the relationship between the external charger and the IPG. Take FIGS. 4A and 4B for example. In FIG. 4A, the coils 52 and 38 in the external charger 50 and the IPG 10 are aligned on the same axis, but the IPG is implanted relatively deeply (d). In FIG. 4B, the coils 52 and 38 are laterally misaligned (x), but the IPG 10 is implanted relatively shallowly. Increasing the depth (d) or the lateral misalignment (x) will reduce the coupling between the coils 52 and 38, which coupling may be the same in both of FIGS. 4A and 4B. Thus, for the same charging current in the external chargers 50 (Icharge=X), the same battery currents result (Ibat2=Y). However, the reality is that the orientation of FIG. 4B (shallow, but misaligned), will dissipate more heat than will the orientation of FIG. 4A (aligned, but deep), and thus in FIG. 4B the IPG 10 generally runs hotter (T>Z) than in FIG. 4A (T=Z). Thus, monitoring Ibat2 alone does not necessarily indicate total IPG heat dissipation.

The two IPGs in FIGS. 4A and 4B dissipate different amounts of heat because more than just the electronic circuitry active in the IPG 10 during charging affects heat dissipation. For example, the conductive case 12 of the IPG 10 is also susceptible to heating during inductive charging due to eddy currents, as mentioned earlier. Eddy current heating can also occur in other conductive structures in the IPG 10. The amount of eddy current heating will vary as the position of the IPG 10 changes relative to the external charger 50; hence the reason that the IPG in FIG. 4B dissipates more heat than the IPG in FIG. 4A. As noted above, one or more thermistors can be used to sense the temperature of the case 12 at any discrete point. But such temperature measurements are not indicative of the total amount of power dissipated as heat.

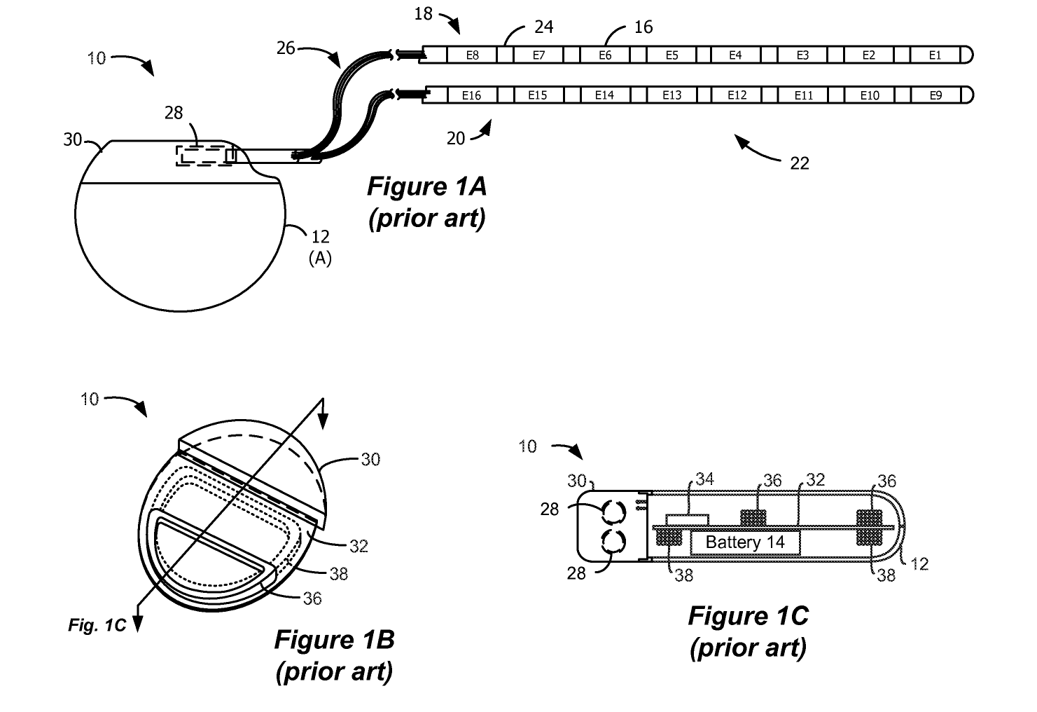

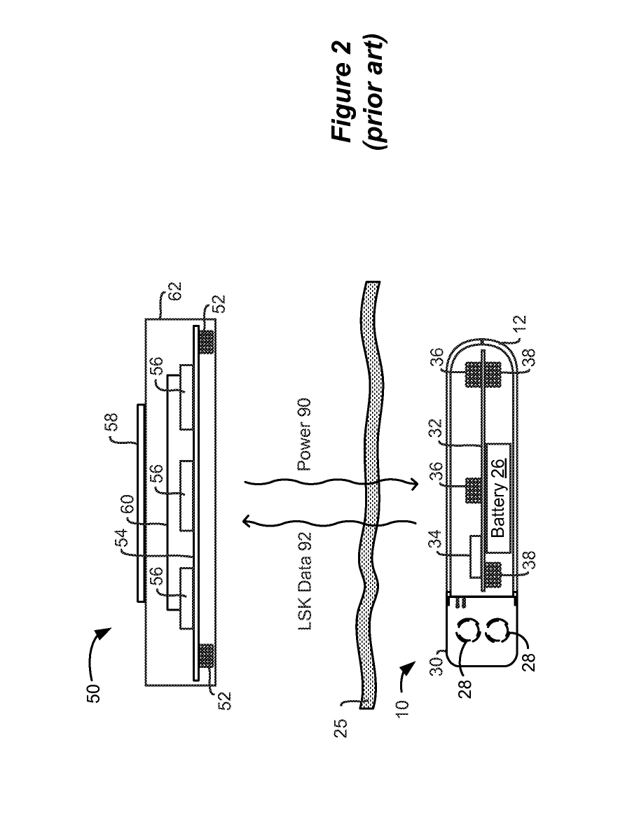

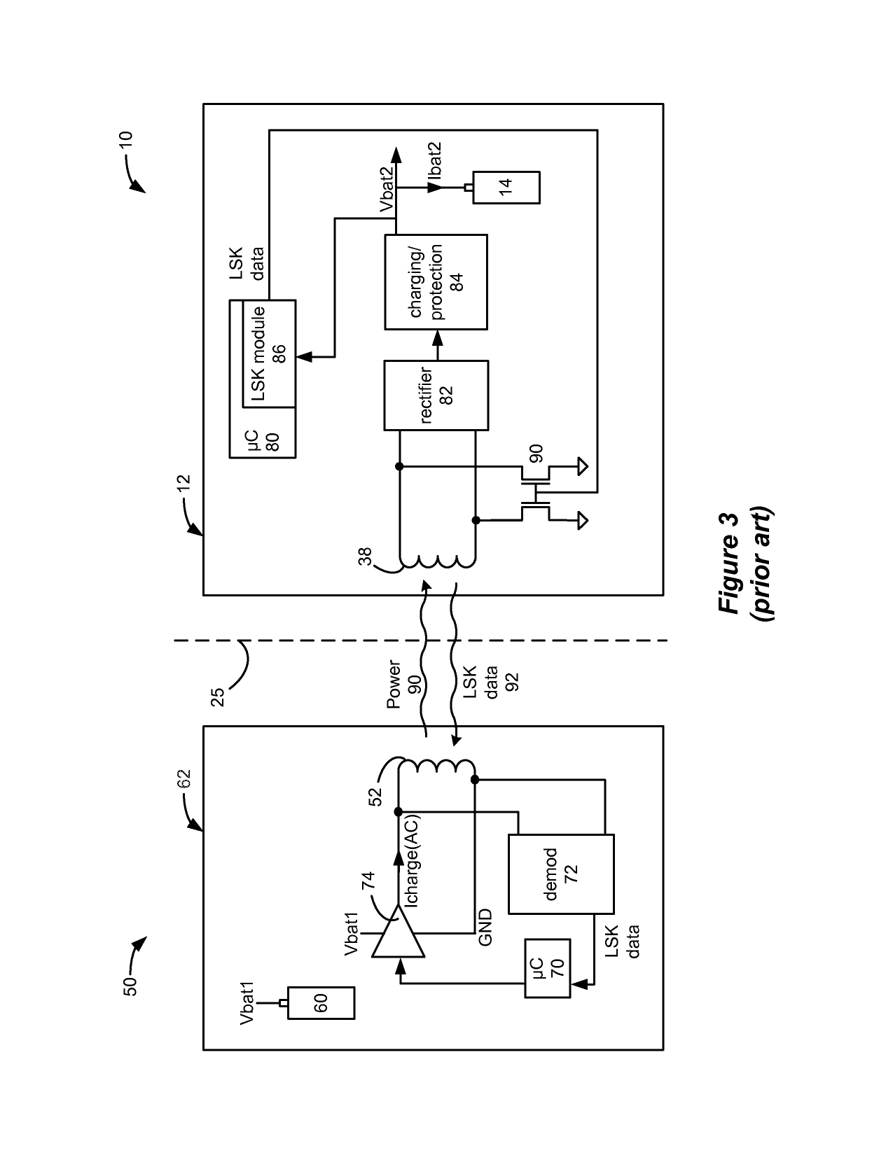

The inability to directly measure the total power dissipated as heat in the IPG during charging is unfortunate, because it is known that safe levels of tissue heating correlate to heat flux, i.e., the amount of heat dissipated from the IPG per unit area. Because the area A of the IPG case 12 (FIG. 1A) is known, if the total power dissipated as heat in the IPG could be measured, then the heat flux could be calculated and compared with known safe heat flux limits.

The disclosed technique measures the total power dissipated as heat in the IPG—albeit indirectly—by accounting for the various powers present in the external charger/IPG system. By way of introduction, the total power provided to the system is measured by measuring the input power provided to the driver circuit 74 (e.g., an amplifier) that drives the coil in the external charger (Psys). To the extent some portion of this system power is coupled to the IPG, that received power is either dissipated in the IPG either as heat (P_IPG) (which the disclosed technique seeks to determine) or stored in the IPG's battery (Pstored) (which is measured and telemetered from the IPG to the external charger). The remaining power not coupled to the IPG returns to the external charger, and is dissipated in the external charger's coil (P_EC) (which is measured). Knowing Psys, Pstored, and P_EC, P_IPG can be calculated at the external charger (P_IPG=Psys−P_EC−Pstored).

Knowing P_IPG and the area A of the IPG's case 12 allows the IPG's heat flux (F_IPG) to be calculated at the external charger, and compared to a safe heat flux limit (F_IPG′). If the external charger determines that the heat flux is above the limit, it reduces the power of the magnetic charging field 90, for example, by reducing its intensity (reducing Icharge) or its duty cycle. Likewise, if below the limit, the external charger can increase the power of the charging field to increase the speed of charging without risk of overheating the implant. In short, the external charger can periodically determine F_IPG and use that parameter to control charging in a closed loop fashion.

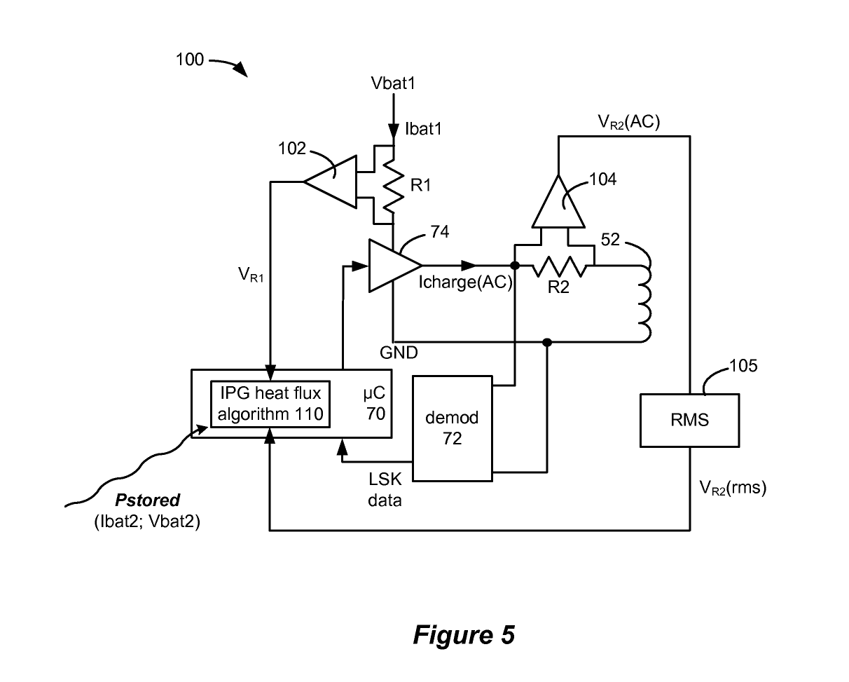

FIG. 5 shows an improved external controller 100 that operates according to the foregoing description to determine the IPG's heat flux, F_IPG, at various points during a charging session and to control charging accordingly in accordance with an algorithm 110. One skilled in the art will understand that algorithm 110 can be programmed into the control circuitry 70 of the external charger 100.

Input to algorithm 110 are signals V_{R1}, indicative of the power provided by the driver circuit 74, and V_{R2}(rms), indicative of the power dissipated by the charging coil 52. These signals comprise voltage drops measured by differential amplifiers 102 and 104 that span resistances R1 and R2 in-line with currents drawn by the driver circuit 74 (Ibat1) and the coil 58 (Icharge) when the external charger 100 is producing a magnetic charging field. V_{R1} and V_{R2}(rms) in effect measure the drawn currents (i.e., Ibat1=V_{R1}/R1; Icharge(rms)=V_{R2}(rms)/R2), which allow the algorithm 110 (knowing R1 and R2) to determine both the power provided to the charging system (Psys=Vbat1*Ibat1), and the fraction of this system power dissipated as heat within the external charger's coil 52 (P_EC). Measurement resistors R1 and R2 are small, on the order of ohms, and hence do not significantly affect the charging operation. Because Icharge is an AC current, note that the output of diff amp 104 (V_{R2}(AC)) has been sent to an RMS measuring circuit 105 to render a DC value V_{R2}(rms).

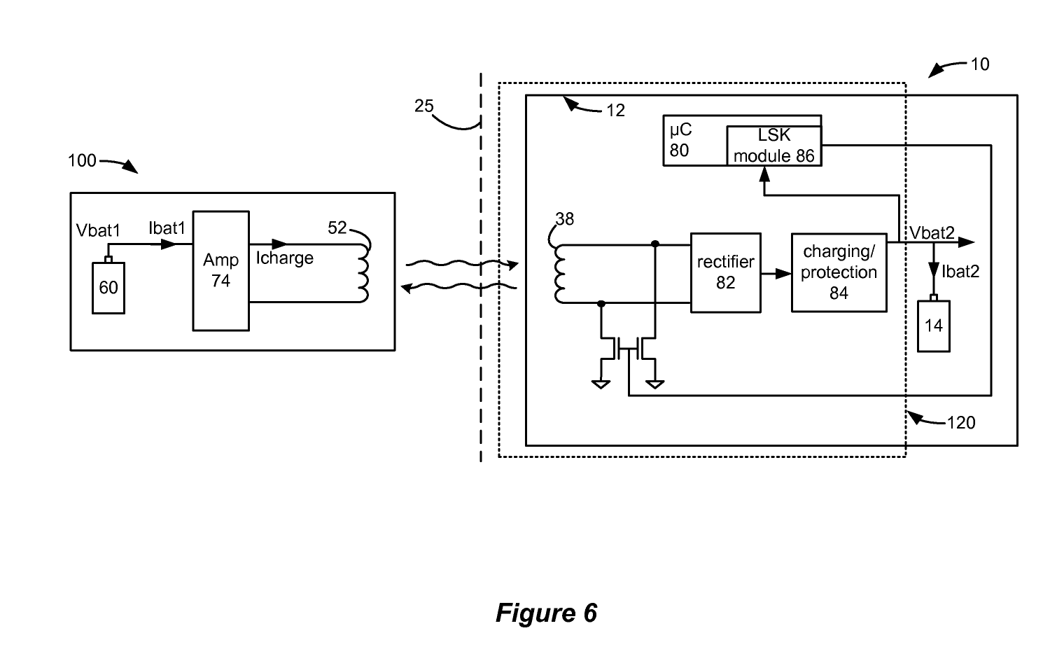

Also input to algorithm 110 is information indicative of the power stored by the IPG's battery 14 (Pstored) during the charging session, which, referring to FIG. 6, equals Vbat2*Ibat2. As discussed further below, such data is provided to the algorithm 110 via telemetry from the IPG 10, which may provide Vbat2 and Ibat2 as separate piece of data, or which may multiply them to provide only a single value for Pstored. One skilled in the art will understand that Vbat2 and Ibat2 are generally monitored in the IPG 10 during a charging operation, and hence circuitry for doing so is known and not further discussed.

Also shown in FIG. 6 are structures 120 that dissipate heat within the IPG 10 during a charging session, including circuits active during a charging session (the control circuit 80, the rectifier 82, the charging/protection circuitry 84, the control circuitry 80, etc.), and the IPG's case 12 and other conductive structures. In short, structures 120 comprise all electrical components (whether active or passive) that dissipate some portion of the power provided by the magnetic charging field 90. As noted above, the power dissipated by the IPG 10 as heat (P_IPG) caused by structures 120 is not directly measured, in particular because the dissipation in the IPG's case 12 and other conductive structures is unknown, but is inferred as explained further below.

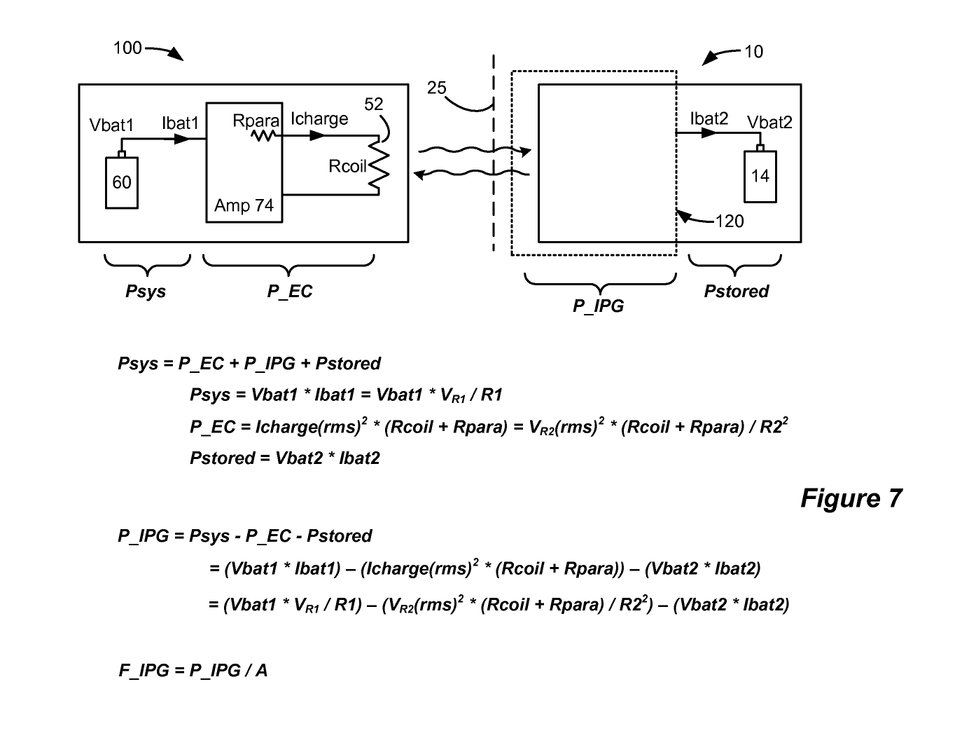

FIG. 7 shows the various power values already introduced in a simplified circuit diagram. The power input to the system (Psys), as already noted, equals the power provided to the driver circuit 74 (Vbat1*Ibat1, or Vbat*V_{R1}/R1), which is used to generate the charging field 90. The fraction of that power ultimately stored in the IPG's battery 14 (Pstored) equals Vbat2*Ibat2. The power dissipated in the external controller 100 as heat (P_EC) includes power from the magnetic charging field that does not couple to the IPG 10; as one skilled in the art understands, such uncoupled power must necessarily return and be dissipated in the external controller's charging coil 52. Because Icharge is an AC current, the power dissipated by the charging coil 52, P_EC equals Icharge(rms)² times the resistance met by this current. This resistance includes the resistance of the charging coil 58 (Rcoil), and also may include parasitic resistances in the output of the driver circuit 74 (Rpara). (If such parasitic resistances are negligible compared to the resistance of the charging coil 52, they may be ignored). Stated in terms of measured value V_{R2}(rms), P_EC=V_{R2}(rms)²*(Rcoil+Rpara)/R2².

This leaves the power dissipated by the structures 120 in the IPG as heat, P_IPG, unaccounted for, but this can be determined by algorithm 110 as follows: the total system power Psys equals the sum of the power dissipated in the external charger 100 (P_EC), the power dissipated in the IPG 10 (P_IPG), and the power stored in the IPG's battery 14 (Pstored), as shown in FIG. 7. Solving for P_IPG, it is noticed that P_IPG is a function of parameters that are: (1) known to the external controller's control circuit 70 (such as Vbat1, which control circuit 70 typically monitors for other reasons); (2) programmable into the control circuit 70 (such as Rcoil, Rpara, R1, and R2); (3) measured in the external charger 100 (such as V_{R1} and V_{R2} (rms)); or (4) telemetered from the IPG 10 (Pstored, or its constituent values Vbat2 and Ibat2). In short, algorithm 110 has the information needed to compute P_IPG.

From P_IPG, the heat flux of the IPG 10, F_IPG can be calculated by dividing it by the area A from which such heat will be dissipated. Because the IPG's header 30 (FIG. 1A) is not significantly heat conducting, this area A is best estimated by the area of the conductive case 12.

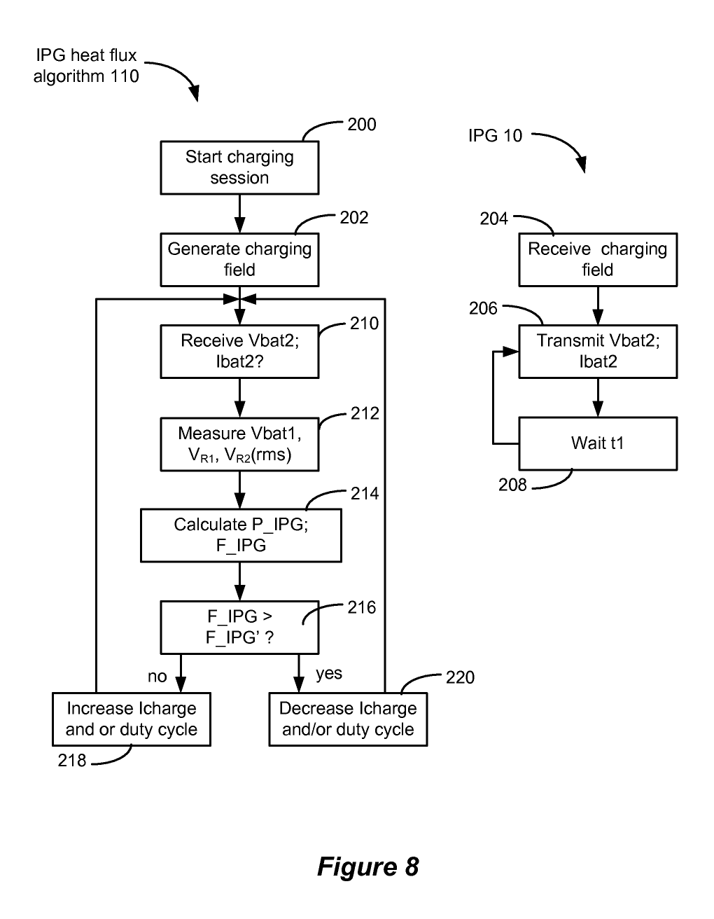

As noted above, heat flux has been correlated to tissue safety, and thus a safe heat flux limit, F_IPG′, can be chosen and stored so that algorithm 110 can control operation of the external charger 100 to charge the IPG's battery 14 without exceeding this heat flux limit. A first example is shown in FIG. 8. In this example, it is assumed that the IPG 10 telemeters Pstored (or its constituent values Vbat2, Ibat2) to the external charger 100 using LSK telemetry. As discussed above, LSK telemetry has the benefit that data can be telemetered from the IPG 10 to the external charger 100 while the magnetic charging field 90 is being generated. In FIG. 8, steps in algorithm 110 in the external charger 100 are shown on the left. Steps taken in the IPG 10 are shown on the right, which horizontally coincide with matching steps taken by the algorithm 110.

Once a charge session has begun (200), and a magnetic charging field 90 generated (202), the IPG 10, upon receipt of this charging field (204), will telemeter Vbat2 and Ibat2 (or Pstored if already calculated) to the external charger 100 (206).

At this point, the IPG 10 can be programmed to wait for a set time (t1) to send its data again (208). This set time t1 determines how often the algorithm 110 will adjust the charging field 90, which for example might range from several seconds to a minute or more.

Upon receipt of Vbat2 and Ibat2 (or Pstored) (210), the algorithm 110 knows that it is now time to measure V_{R1} and V_{R2}(rms) (212), and may also measure Vbat1 if not already known. The algorithm may therefore enable differential amplifiers 102 and 104 at these times to receive these values.

The algorithm 110 now has the data necessary (in addition to Vbat1, and the various resistances discussed above) to calculate P_IPG and F_IPG (214). The algorithm 110 compares F_IPG to limit F_IPG′ (216) to decide how to modify the magnetic charging field 90. If F_IPG>F_IPG′, the power in the magnetic charging field is reduced (220), which can occur by decreasing Icharge, or the duty cycle of the field. If F_IPG<F_IPG′, this may indicate that the power of the charging field can be safely increased (218), thus allowing the IPG's battery 14 to charging more quickly.

At this point, the external charger 100 again waits for receipt of telemetry from the IPG 10 (210), which again will not occur until after expiration of the set time t1 (208). Upon receipt of new data from the IPG 10, the algorithm can once again measure (212) and calculate (214) to adjust the magnetic charging field 90 in a closed loop fashion.

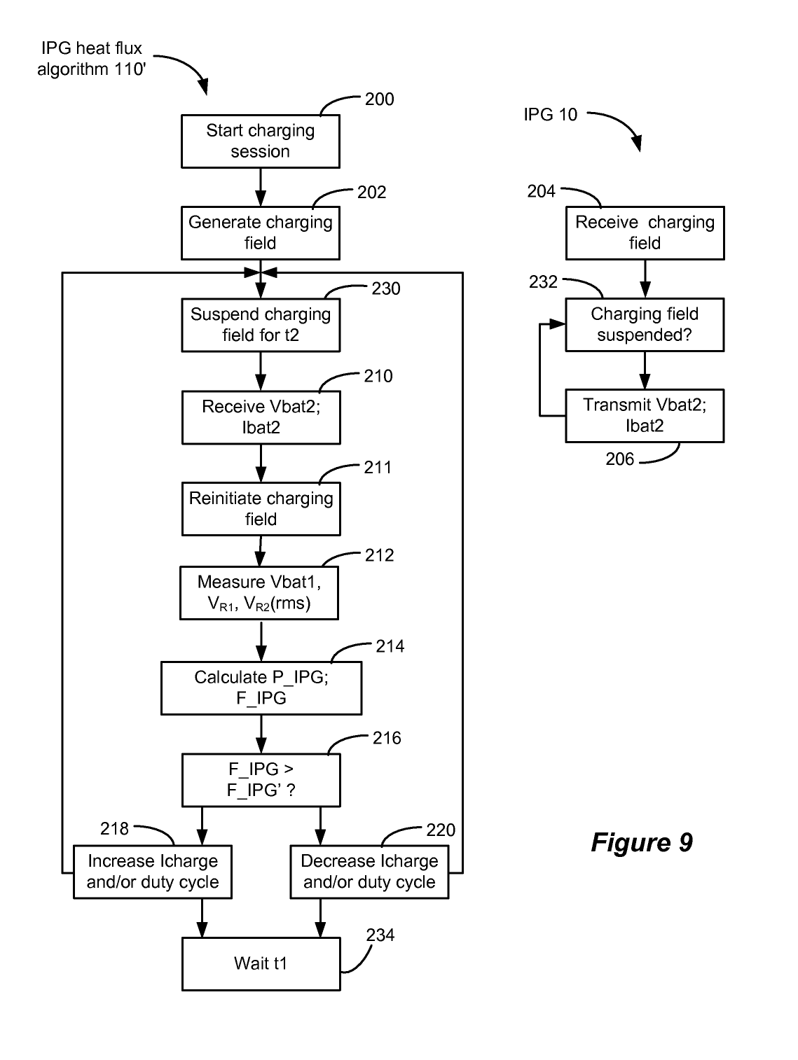

FIG. 9 shows another example of algorithm 110′. Many of the steps in FIG. 9 are the same as in FIG. 8, and are similarly labeled and not discussed again in detail.

In algorithm 110′, the IPG 10 communicates with the external charger 100 using a different means of telemetry aside from LSK. As one skilled in the art will recognize, such alternative means of communication can occur in any number of ways. For example, the IPG 10 can use its data telemetry coil 36, which it normally uses to communicate with some other external controlling device. Typically, the data telemetry coil 36 communicates using a Frequency Shift Keying (FSK) protocol. Such data can be received by the charging coil 52 in the external charger 52, although it may need to be re-tuned to the center frequency of such FSK communications. See, e.g., U.S. Patent Application Publication 2011/0112611. The external charger 100 could also be modified to include another coil to communicate with the IPG 10. Alternatively, both the IPG 10 and the external charger 100 can include RF antennas to communicate using known short-range RF protocols, such as Bluetooth, MICS, Zigbee, etc.

Because it may be difficult to telemeter data from the IPG 10 to the external charger while the magnetic charging field 90 is active, the charging field is temporarily suspended in algorithm 110′ for a short time t2 to allow data transfer to occur (230), which may be significantly less than a second. The IPG 10, upon noticing that the charging field has been suspended (232), can understand that it needs to transmit Vbat2 and Ibat2 (or Pstored) (206). Afterwards, the IPG 10 can simply wait for the charge field to cease again.

Upon receipt of the IPG's data (210), the algorithm 110′ can reinitiate the charging field (211), and as before, measure (212) and calculate (214) to determine P_IPG and F_IPG (214), and compare F_IPG to the limit (216) to either increase (218) or decrease (220) the power the magnetic charging field 90. At this time, the algorithm 110′ can wait for a set time t1 (234) before suspending the magnetic charging field again (230). As before t1 determines how often the algorithm 110 will adjust the charging field. At this point, the IPG will send new data (206, 210), and the algorithm 110′ can once again measure (212) and calculate (214) to adjust the magnetic charging field 90 in a closed loop fashion.

Other modifications to the disclosed system and methods are possible. For example, it has been disclosed that Psys is determined with reference to the power input to the driver circuit 74 that energizes the coil 52 to produce the magnetic charging field 90. This is considered desirable because it allows the algorithm 110 to ignore other power drains in the external charger 100 that can be considered “overhead” in producing the magnetic charging field, such as power drawn by the control circuitry 70, the demodulator 72, and other circuits that have not been depicted for simplicity. However, one could also define and measure the input power Psys as comprising the total power provided by the external charger's battery 60 to all circuits in the external charger 100. In such a case, it would be necessary for algorithm 110 to measure or estimate the power drawn by such overhead circuitry (P_overhead) in the external charger, so it could be subtracted out of the algorithm's calculation when determining P_IPG and F_IPG.

Furthermore, while the disclosed technique has centered around making charging adjustments in light of the IPG's heat flux, it should be noted that the technique is not so limited. In other useful embodiments, determinations made based on the power dissipated in the IPG, P_IPG, could be used as well. One skilled will also realize that there are different ways the parameters disclosed herein could be measured, processed, telemetered, etc.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims. cm What is claimed is:

1. A method of controlling an external charger for an implantable medical device, comprising:
determining at the external charger a first power input to the external charger as it energizes a coil of the external charger to produce a charging field for storing charge in a battery in the implantable medical device;
determining at the external charger a second power dissipated in the energized coil;
determining at the external charger a third power stored in the battery of the implantable medical device; and
controlling the charging field using at least the first, second, and third powers.

2. The method of claim 1, wherein the external charger comprises a driver circuit for energizing the coil, and wherein the first power comprises a power input to the driver circuit as it energizes the coil.

3. The method of claim 2, wherein the first power comprises a voltage supplied to the driver circuit times a current drawn by the driver circuit.

4. The method of claim 2, wherein determining the first power comprises measuring a value indicative of a current drawn by the driver circuit.

5. The method of claim 1, wherein determining the second power comprises measuring a value indicative of a current drawn by the coil.

6. The method of claim 5, wherein the value comprises an RMS value.

7. The method of claim 1, wherein determining the third power comprises receiving data from the implantable medical device by telemetry.

8. The method of claim 7, wherein the data comprises a current drawn by the battery and a voltage across the battery, or the product of the current drawn by the battery and the voltage across the battery.

9. The method of claim 7, wherein the data is received at the coil.

10. The method of claim 7, wherein the charging field is temporarily suspended when receiving the data from the implantable medical device.

11. The method of claim 7, wherein the data is received via reflected impedance modulation of the charging field.

12. The method of claim 1, wherein controlling the charging field using at least the first, second, and third powers comprises determining a fourth power by subtracting the second and third powers from the first power.

13. The method of claim 12, wherein the charging field is controlled using the fourth power.

14. The method of claim 1, wherein the first, second, and third powers are used to compute a heat flux of the implantable medical device, and wherein the charging field is controlled using the computed heat flux.

15. The method of claim 14, wherein the computed heat flux is compared to a heat flux limit.

16. The method of claim 15, wherein if the computed heat flux is greater than the heat flux limit, the charging field is controlled by reducing a power of the charging field.

17. The method of claim 16, wherein the power of the charging field is reduced by reducing a current of the coil or by reducing a duty cycle of the charging field.

18. An external charger for an implantable medical device, comprising:
a driver circuit configured to provide a charge current to a coil to produce a magnetic charging field to store charge in a battery in the implantable medical device; and
a control circuit configured to control the driver circuit in accordance with an algorithm configured to determine a power dissipated in the implantable medical device,
wherein the algorithm determines the power dissipated in the implantable medical device by subtracting a power stored in the battery and power dissipated in the coil from a power input to the driver circuit.

19. The external charger of claim 18, further comprising a first amplifier configured to measure a current drawn by the driver circuit.

20. The external charger of claim 19, wherein the power input to the driver circuit comprises the current drawn by the driver circuit times a voltage supplied to the driver circuit.

21. The external charger of claim 19, further comprising a second amplifier configured to measure a current drawn by the coil.

22. The external charger of claim 21, wherein the power dissipated in the coil comprises the current drawn by the coil squared, times a resistance of the coil.

23. The external charger of claim 22, wherein the current drawn by the coil comprises an RMS value.

24. The external charger of claim 18, further comprising a demodulation circuit configured to receive data from the implantable medical device.

25. The external charger of claim 24, wherein the data is indicative of the power stored in the battery.

26. The external charger of claim 24, wherein the demodulation circuit is coupled to the coil.

27. The external charger of claim 26, wherein the demodulation circuit is configured to receive the data via reflected impedance modulation of the charging field.

28. The external charger of claim 24, wherein the control circuit is configured to temporarily suspend the driver circuit when the demodulation circuit is configured to receiving the data from the implantable medical device.

29. The external charger of claim 18, wherein the driver circuit is controlled using the power dissipated in the implantable medical device.

30. The external charger of claim 18, wherein the driver circuit is controlled using a heat flux of the implantable medical device, and wherein the heat flux of the implantable medical device is computed using the power dissipated in the implantable medical device.

31. The external charger of claim 30, wherein the driver circuit is controlled by comparing the computed heat flux to a heat flux limit.

32. The external charger of claim 31, wherein if the computed heat flux is greater than the heat flux limit, the control circuit is configured to reduce a power of the charging field.

33. The external charger claim 32, wherein the control circuit is configured to reduce the power of the charging field by reducing a current of the coil or by reducing a duty cycle of the charging field.
